(19) United States
(12) Patent Application Publication
(10) Pub. No.: US 2015/0086481 A1
(43) Pub. Date: Mar. 26, 2015

(54) DIAGNOSIS AND TREATMENT OF PARKINSON'S DISEASE

(71) Applicants: Yosif Ganat, New York, NY (US); Lorenz Studer, New York, NY (US)

(72) Inventors: Yosif Ganat, New York, NY (US); Lorenz Studer, New York, NY (US)

(73) Assignee: MEMORIAL SLOAN-KETTERING CANCER CENTER, New York, NY (US)

(21) Appl. No.: 14/235,534

(22) PCT Filed: Jul. 27, 2012
(86) PCT No.: PCT/US2012/048621

§ 371 Date: Apr. 25, 2014

Related U.S. Application Data
(60) Provisional application No. 61/574,166, filed on Jul. 28, 2011.

(51) Int. Cl. A61K 47/48; A61K 31/197; A61K 51/08

(52) U.S. Cl. CPC A61K 47/48246; A61K 51/088; A61K 31/197
USPC 424/1.69; 530/326; 514/17.7

(57) ABSTRACT

GUCY2C receptor ligands conjugated to diagnostic and/or therapeutic moieties, methods of making such GUCY2C receptor ligands conjugates, and methods of using such GUCY2C receptor ligands conjugates, such as in the diagnosis and/or treatment of Parkinson's disease, are described.

### CROSS REFERENCE TO RELATED APPLICATIONS

This application claims the benefit of U.S. Provisional Application No. 61/574,166, filed Jul. 28, 2011, the contents of which are hereby incorporated herein in their entirety.

### BACKGROUND

Parkinson's disease (PD) is characterized by the death of midbrain dopaminergic (mDA) neurons in the substantia nigra (SN), which results in severe motor deficits and dyskinesias. Despite affecting nearly 1 million people in the US, no standardized method for diagnosing PD exists. PD is further complicated by the fact that when a patient begins to develop motor deficits they have typically lost 40-50% of their mDA neuron population. Thus, early diagnosis is important for PD intervention strategies.

### SUMMARY

The present disclosure provides the discovery that GUCY2C is expressed in the brain specifically by midbrain dopamine neurons in the SN. The present disclosure also provides the discovery that guanylin, a GUCY2C receptor ligand, selectively targets GUCY2C on mDA neurons. The present invention therefore provides GUCY2C receptor ligands conjugated to detectable and/or therapeutic moieties, and the use of such ligands in the diagnosis and/or treatment of Parkinson's disease.

In some aspects, the invention features a composition, e.g., a diagnostic and/or a therapeutic composition, comprising a GUCY2C receptor ligand conjugate comprising or consisting of a GUCY2C receptor ligand conjugated to a payload moiety. In some embodiments, upon providing (e.g., administering) the composition to a subject, the GUCY2C receptor ligand conjugate crosses the blood brain barrier (BBB) of the subject. In some embodiments, upon administering the composition to the subject, at least about 20%, 25%, 30%, 35%, 40%, 45%, 50%, 55%, 60%, 65%, 70%, 75%, 80%, 85%, 90%, 95%, or 100%, of the GUCY2C receptor ligand conjugate crosses the BBB. In some embodiments, the payload moiety is a detectable moiety. In some embodiments, the payload moiety is a therapeutic moiety.

In some aspects, the invention features a method of identifying a subject having or at risk of developing Parkinson's disease, the method comprising: providing (e.g., administering) to a subject a GUCY2C receptor ligand conjugate comprising or consisting of a GUCY2C receptor ligand conjugated to a detectable moiety, wherein the GUCY2C receptor ligand conjugate crosses the blood brain barrier of the subject. In some embodiments, the method further comprises measuring a level of the detectable moiety bound to neurons in the midbrain (e.g., midbrain dopamine neurons) of the subject, wherein a level of detectable moiety bound to the neurons less than a predetermined level indicates the subject has or is at risk of developing Parkinson's disease. In some embodiments, a predetermined level is a level of detectable moiety measured bound to neurons in the midbrain (e.g., midbrain dopamine neurons) of a control subject, e.g., a subject not having or not at risk of developing PD. In some embodiments, the method further comprises selecting the subject for treatment of Parkinson's disease if the level of detectable moiety bound to neurons is less than the predetermined level. In some embodiments, the method further comprises administering a therapeutic agent to the subject if the level of detectable moiety bound to neurons is less than the predetermined level. In some embodiments, the level of the detectable moiety bound to the neurons is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, less than the predetermined level.

In some aspects, the invention features a method of treating a subject having or at risk of developing Parkinson's disease, comprising: providing (e.g., administering) to the subject a GUCY2C receptor ligand conjugate comprising or consisting of a GUCY2C receptor ligand conjugated to a therapeutic moiety, wherein the GUCY2C receptor ligand conjugate crosses the blood brain barrier of the subject, thereby treating the subject.

In some aspects, the invention features a method of identifying a targeting moiety that binds to midbrain neurons of a subject, comprising: providing (e.g., administering) to a subject a GUCY2C receptor ligand conjugate comprising or consisting of a GUCY2C receptor ligand conjugated to a detectable moiety, wherein the GUCY2C receptor ligand conjugate crosses the blood brain barrier of the subject; measuring a first level of the detectable moiety bound to neurons in the midbrain of the subject; providing (e.g., administering) to the subject a test moiety; and measuring a second level of the detectable moiety bound to the neurons in the midbrain of the subject, wherein the test moiety is identified as a targeting moiety if the second level is lower than the first level.

In some embodiments of any of the aspects described herein, the GUCY2C receptor ligand is guanylin. In some embodiments of any of the aspects described herein, the detectable moiety is ¹²⁴I. In some embodiments of any of the aspects described herein, measuring the level of the detectable moiety comprises positron emission tomography (PET).

### BRIEF DESCRIPTION OF THE DRAWINGS

The following figures are presented for the purpose of illustration only, and are not intended to be limiting.

FIG. 1A is a Venn diagram plot for significantly enriched (increased expression only) genes in intraline analysis, showing 107 genes common to both Nurr1::GFP+ and Pitx3::YFP+. FIG. 1B is a Venn diagram plot of significantly enriched genes in interline analysis, showing 148 genes common to both Nurr1::GFP+ and Pitx3::YFP+ cells in contrast to Hes5::GFP+ cells. FIGS. 1C-lE are scatter plots of significantly altered genes (≧2 fold increased/decreased; P≦0.05). Light gray boxes represent genes enriched at the Hes5 stage (H+/H−), while dark gray boxes represent genes that are decreased at that stage. FIG. 1C is an intraline scatter plot of the 233 altered H+/H− genes (x axis) compared with the altered 232 N+/N− genes (y axis) (449 genes common to both). FIG. 1D is an intraline scatter plot of the 232 altered N+/N− genes compared with the 556 altered P+/P− genes (656 genes common to both). FIG. 1E is an interline scatter plot of the 586 altered N+/H+ genes compared with the 1,203 altered P+/H+ genes (1,355 genes common to both).

FIG. 2A is a representation of a sagittal slice of a brain in the Allen brain atlas© showing GUCY2C expression. FIG. 2B is a representation of a sagittal slice of a brain in the GENSAT database showing GUCY2C expression.

FIG. 3 is a representation of autoradiography of a 50 μM sagittal section of adult mouse brain after tail vein injection of ¹³¹I-guanylin showing a strong emission in the ventricles and SN, indicating specific binding to the GUCY2C receptor.

FIG. 4A is a representation of immunohistochemical analysis of GUCY2C expression in the 6-8 week human fetal midbrain. Expression of GUCY2C included the Lmx1a and TH mDA neuron progenitor domain, as well as the broader domain to which these cells migrate and mature. FIG. 4B is a representation of an image of Allen brain atlas Brain Explorer data demonstrating strong GUCY2C mRNA expression in the adult SN.

All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Other features and advantages of the invention will be apparent from the following detailed description, and from the claims.

### DEFINITIONS

In order for the present invention to be more readily understood, certain terms are first defined below. Additional definitions for the following terms and other terms are set forth throughout the specification.

Amelioration: As used herein, the term “amelioration” means the prevention, reduction or palliation of a state, or improvement of the state of a subject. Amelioration includes, but does not require, complete recovery or complete prevention of a disease condition.

Amino acid entity: As used herein, an “amino acid entity” is any compound or substance that can be incorporated into a polypeptide chain without terminating or significantly disrupting a polypeptide chain. In some embodiments, an amino acid entity is incorporated into a polypeptide chain through participation in one or more peptide bonds. In some embodiments, an amino acid entity is a naturally-occurring amino acid (e.g., arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine). In some embodiments, an amino acid entity is a synthetic amino acid entity. In some embodiments, an amino acid entity is a D-amino acid. In some embodiments, an amino acid entity is an L-amino acid. In some embodiments, an amino acid entity is a modified, e.g., chemically modified, amino acid. In some embodiments, an amino acid entity comprises one or more additional methyl, amino, or acetyl groups as compared with a reference amino acid (e.g., a naturally-occurring amino acid).

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Characteristic portion: As used herein, the term a “characteristic portion” of a substance, in the broadest sense, is one that shares some degree of sequence or structural identity with respect to the whole substance. In certain embodiments, a characteristic portion shares at least one functional characteristic with the intact substance. For example, in some embodiments, a “characteristic portion” of a polypeptide or protein is one that contains a continuous stretch of amino acids, or a collection of continuous stretches of amino acids, that together are characteristic of a polypeptide or protein. In some embodiments, each such continuous stretch generally contains at least 2, 5, 10, 15, 20, 50, or more amino acids. In some embodiments, such a continuous stretch includes certain residues whose position and identity are fixed; certain residues whose identity tolerates some variability (i.e., one of a few specified residues is accepted); and optionally certain residues whose identity is variable (i.e., any residue is accepted). In general, a characteristic portion of a substance (e.g., of a polypeptide or protein) is one that, in addition to the sequence and/or structural identity specified above, shares at least one functional characteristic with the relevant intact substance. In some embodiments, a characteristic portion may be biologically active.

Characteristic sequence: A “characteristic sequence” is a sequence that is found in all members of a family of polypeptides or nucleic acids, and therefore can be used by those of ordinary skill in the art to define members of the family.

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

Comparable: The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

Correlates: The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

Detectable moiety: As used herein, a “detectable moiety” refers to a molecular structure or module that allows visualization, imaging, measurements (localization, quantification, etc.) and/or monitoring of an agent in vitro and/or in vivo using one or more detection techniques including but not limited to spectroscopic, photochemical, biochemical, immunochemical, electrical, optical, chemical, scintillation, or other means.

Fluorophore: As used herein, terms “fluorophore,” “fluorescent moiety,” “fluorescent label,” “fluorescent dye” and “fluorescent labeling moiety” are used herein interchangeably. They refer to a molecule that, in solution and upon excitation with light of appropriate wavelength, emits light. Numerous fluorescent dyes of a wide variety of structures and characteristics are suitable for use in the methods of the disclosure. In some embodiments, a fluorophore absorbs light and emits fluorescence with high efficiency (i.e., high molar absorption coefficient and fluorescence quantum yield, respectively) and/or is photostable (i.e., it does not undergo significant degradation upon light excitation within the time necessary to perform the analysis). Fluorophores useful in the disclosed embodiments may be selected for advantageous diagnostic features such as wavelength (e.g., near infrared or infrared) that produces a high signal to noise ratio when used in vivo.

GUCY2C ligand: As used herein, the term “GUCY2C ligand” refers to an entity that specifically binds to a GUCY2C receptor. In some embodiments, a GUCY2C ligand is a polypeptide, a small molecule, or a nucleic acid. In some embodiments, a GUCY2C is a natural, a synthetic, or a recombinant GUCY2C ligand. In some embodiments, a GUCY2C ligand is a polypeptide whose amino acid sequence includes at least one characteristic sequence of and/or shows at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71% or 70% amino acid identity with a GUCY2C ligand described herein. In some embodiments, a GUCY2C ligand shares at least one characteristic sequence of and/or shows the specified degree of overall sequence identity with SEQ ID NO:1 or SEQ ID NO:2 (which may be considered a “reference” GUCY2C ligand). In some embodiments, a GUCY2C ligand is an agonist that binds to a GUCY2C receptor and increases an observed signaling activity of the GUCY2C receptor relative to that of the GUCY2C receptor in an unliganded (unbound) state. In some embodiments, a GUCY2C ligand is an antagonist that binds to a GUCY2C receptor and decreases an observed signaling activity of the GUCY2C receptor relative to that observed upon binding of an agonist of the GUCY2C receptor to the GUCY2C receptor.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, or reduce: As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein.

Polypeptide: As used herein, a “polypeptide” is a polymer comprising at least two amino acid entities attached to one another by a peptide bond. In some embodiments, a polypeptide includes at least 3-5 amino acid entities, each of which is attached to others by way of at least one peptide bond. In some embodiments, a polypeptide is or comprises a prepropolypeptide, propolypeptide or prepolypeptide. In some embodiments, a polypeptide is or comprises a polypeptide chain having an amino acid sequence identical to that of a protein that is naturally produced by a cell. In some embodiments, a polypeptide is or comprises a polypeptide chain having an amino acid sequence that is not identical to that of any protein known to be produced by a cell as of the effective date of the present disclosure. In some embodiments, a polypeptide is produced by a cell. In some embodiments, a polypeptide is produced using chemical synthesis.

Providing: As used herein, the term “providing” refers to performing a manipulation that causes an entity of interest to be present at a level and/or with an activity higher than that observed under otherwise comparable conditions prior to or absent the manipulation. In some embodiments, providing consists of or comprises administering the entity itself (alone or as part of a composition); in some embodiment, providing consists of or comprises administering an agent that causes an increase in level and/or activity of the entity of interest. For example, where the entity of interest is or comprises a polypeptide, in some embodiments, “providing” the polypeptide consists of or comprises administering the polypeptide (e.g., to a cell, whether isolated or in an organism); in some embodiments, “providing” the polypeptide consists of or comprises administering a nucleic acid encoding the polypeptide; in some embodiments, “providing” the polypeptide consists of or comprises administering an agent that results in increased expression of an endogenous copy of the polypeptide (e.g., by stimulating one or more of transcription, RNA processing, translation, etc. and/or by inhibiting an inhibitor of one of these).

Reference: A “reference” entity, system, amount, set of conditions, etc., is one against which a test entity, system, amount, set of conditions, etc. is compared as described herein. For example, in some embodiments, a “reference” individual is a control individual who is not suffering from or susceptible to any form of Parkinson's disease; in some embodiments, a “reference” individual is a control individual afflicted with the same form and/or degree of Parkinson's disease as an individual being treated, and optionally who is about the same age as the individual being treated (to ensure that the stages of the disease in the treated individual and the control individual(s) are comparable).

Subject: As used herein, the term “subject”, “individual”, or “patient” refers to any organism upon which embodiments of the invention may be used or administered, e.g., for experimental, diagnostic, prophylactic, and/or therapeutic purposes. In some embodiments, a subject is a mammal, e.g., a human or non-human primate (e.g., an ape, monkey, orangutan, or chimpanzee), a dog, cat, guinea pig, rabbit, rat, mouse, horse, cattle, or cow.

Target cell or target tissue: As used herein, the term “target cell” or “target tissue” refers to any cell, tissue, or organism that is affected by Parkinson's disease to be treated, or any cell, tissue, or organism in which a protein involved in Parkinson's disease is expressed. In some embodiments, target cells, target tissues, or target organisms include those cells, tissues, or organisms in which there is a detectable or abnormally low amount of GUCY2C polypeptide (e.g., comparable to that observed in patients not suffering from or susceptible to Parkinson's disease). In some embodiments, target cells, target tissues, or target organisms include those cells, tissues, or organisms that display a disease-associated pathology, symptom, or feature.

Therapeutic agent: As used herein, the phrase “therapeutic agent” (or “therapeutic moiety”) refers to any agent that, when administered to a subject, has a therapeutic effect and/or elicits a desired biological and/or pharmacological effect.

Therapeutic regimen: As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease such as Parkinson's disease. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g., transcription, processing, and/or translation of a particular gene product, such as a primary transcript or mRNA).

Therapeutically effective amount: As used herein, the term “therapeutically effective amount” refers to an amount of a therapeutic agent (e.g., neuroprotective agent) which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the severity of the Parkinson's disease; the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

Treatment: As used herein, the term “treatment” (also “treat” or “treating”) refers to any administration of a therapeutic agent (e.g., a neuroprotective agent) according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., Parkinson's disease); in some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION

The present disclosure encompasses the discovery that a GUCY2C receptor ligand, guanylin, can be administered to a subject to target GUCY2C receptors on midbrain dopamine (mDA) neurons. Parkinson's disease (PD) is characterized by a loss of mDA neurons within the substantia nigra, leading to debilitating motor defects. Accordingly, the disclosure provides, among other things, various diagnostic and therapeutic modalities, including use of GUCY2C receptor ligands to treat and/or diagnose PD.

### Parkinson's Disease

Parkinson's disease (PD) is a major neurodegenerative disease characterized by muscle rigidity, tremor, and bradykinesia (Dunnett and Bjorklund, Nature 399:A32-A39 (1999)). Other symptoms such as postural deficits, gait impairment, and dementia are also observed in a subpopulation of PD patients. Although the majority of idiopathic PD cases are sporadic and probably influenced by environmental factors, familial aggregation of cases and rare mendelian inheritance of PD traits evince the importance of genetics.

Parkinsonism is a clinical syndrome primarily involving the following symptoms: tremor at rest, bradykinesia, a decrease in spontaneity and movement, rigidity, and postural instability. Less prominent manifestations involve changes in mood and intellect, autonomic function, and the sensory system. The average age at onset is 55 years, with about 1% of persons 60 years of age or older having the disease. Men are affected more frequently than women.

Resting tremor and bradykinesia are the most typical parkinsonian signs. Bradykinesia accounts for most of the associated parkinsonian symptoms and signs: general slowing down of movements and of activities of daily living; lack of facial expression (hypomimia or masked facies); staring expression due to decreased frequency of blinking; impaired swallowing, which causes drooling; hypokinetic and hypophonic dysarthria; monotonous speech; small handwriting (micrographia); difficulties with repetitive and simultaneous movements; difficulty in arising from chair and turning over in bed; shuffling gait with short steps; decreased arm swing and other automatic movements; and start hesitation and freezing. Freezing, manifested by sudden and often unpredictable inability to move, is one of the most disabling of all parkinsonian symptoms.

In Parkinson's disease (PD), the level of dopamine is decreased in the striatum, but most severely in the putamen. This is largely a result of degeneration of dopamine-producing neurons in the substantia nigra pars compacta (Yamada et al., Brain Res. 526:303-307 (1990); Damier et al., Brain 122:1437-1448 (1999); and Naoi et al., Mech. Ageing Dev. 111:175-188 (1999)). Three genes have been associated with autosomal dominant PD, NR4A2 (Le et al., Nat. Genet. 33:85-89 (2003), α-synuclein (Polymeropoulos et al., Science 276:2045-2047 (1997), and ubiquitin C-terminal hydroxylase L1 (UCHL1) (Wintermeyer et al., Neuroreport 11:2079-2082 (2000). The two genes that have been associated with autosomal recessive PD are parkin (Kitada et al., Nature 392:605-608 (1998)) and DJ-1 (Bonifati et al., Science 299:256-259 (2003)). Inactivating mutations of the parkin gene cause PARK2 autosomal recessive juvenile parkinsonism (AR-JP). Similar to other PD forms, PARK2 is characterized by loss of dopaminergic neurons in the substantia nigra. However, PARK2 is unique in that Lewy bodies in substantia nigra neurons are absent in most cases of AR-JP (Ishikawa and Tsuji, Neurology 47:160-166 (1996); Ishikawa and Takahashi, J. Neurol. 245:4-9 (1998); and Matsumine, J. Neurol. 245:10-14 (1998)). Mutations in the parkin gene cause a from of AR-JP but are also found in older PD patients, demonstrating that parkin mutations are not limited to juvenile onset (Abbas et al., Hum. Mol. Genet. 8:567-574 (1999)).

### GUCY2C Receptor and Ligands

The present disclosure encompasses the finding that GUCY2C is expressed in the brain by midbrain dopamine (mDA) neurons. Guanylyl cyclase C (GCC, also referred to as GUCY2C) is an intestinal tumor suppressor, and the nucleic acid and amino acid sequences are known (see, e.g., de Sauvage et al., J. Biol. Chem. 266:17912 (1991)). GUCY2C is principally expressed in intestinal epithelial cells (Carrithers et al., Proc. Natl. Acad. Sci. U.S.A. 93(25):14827-14832 (1996); Carrithers et al., Gastroenterology 107:1653-1661 (1994); Swenson et al., Biochem. Biophys. Res. Commun. 225(3):1009-1014 (1996). GUCY2C is the receptor for diarrheagenic bacterial enterotoxins (STs) (Schulz et al., Cell 63:941-948 (1990)) and the gut paracrine hormones, guanylin (Currie et al., Proc. Natl. Acad. Sci. U.S.A. 89:947-951 (1992)), and uroguanylin (Hamra et al., Proc. Natl. Acad. Sci. U.S.A. 90:10464-10468 (1993)). These ligands regulate water and electrolyte transport in the intestinal and renal epithelia, and are ultimately responsible for acute secretory diarrhea.

Guanylin (GUCA2A) is a polypeptide of 15 amino acids in length having the amino acid sequence PNTCEICAYAACTGC (SEQ ID NO:1). Uroguanylin (GUCA2B) is a polypeptide of 16 amino acids in length having the amino acid sequence QEDCELCINVACTGC (SEQ ID NO:2). They are both secreted by intestinal epithelial cells as prohormones, and are converted enzymatically into active hormones. According to the present disclosure, such polypeptide ligands are useful in the diagnosis and treatment of PD.

In some embodiments, such GUCY2C polypeptide ligands useful in the practice of the present disclosure have or include amino acid sequences as set forth in SEQ ID NO:1 or 2, or characteristic sequence elements thereof or therein. In some embodiments, useful GUCY2C polypeptide ligands show at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% overall sequence identity with SEQ ID NO:1 or 2. Alternatively or additionally, in some embodiments, useful polypeptide ligands include at least about 4, 5, 6, 7, 8, 9, 10, 11, 12, 13, 14, 15, or 16 contiguous amino acids found in SEQ ID NO:1 or 2.

In some embodiments, a useful GUCY2C polypeptide ligand differs from its reference ligand (e.g., a polypeptide having or including an amino acid sequence as set forth in SEQ ID NO:1 or 2, or characteristic sequence elements thereof or therein) by one or more amino acid residues. For example, in some embodiments, the difference is a conservative or nonconservative substitution of one or more amino acid residues. Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

In some embodiments, useful GUCY2C polypeptide ligands are conjugated to (e.g., fused, linked, coupled to, or labeled with) a payload moiety (e.g., a detectable moiety and/or a therapeutic moiety described herein). In some embodiments, useful GUCY2C polypeptide ligands are fused, linked, or coupled to an amino acid sequence (e.g., a leader sequence, a secretory sequence, a proprotein sequence, a second polypeptide, or a sequence that facilitates purification, enrichment, or stabilization of the polypeptide).

In some embodiments, a GUCY2C ligand is an agonist of a GUCY2C receptor. In some embodiments, a GUCY2C ligand is an antagonist of a GUCY2C receptor.

In some embodiments, a GUCY2C ligand binds to a GUCY2C receptor with a binding affinity of Kₐ less than or equal to about 10⁻⁷, 10⁻⁸, 10⁻⁹, 10⁻¹⁰, 10⁻¹¹ or 10⁻¹² M.

### Methods of Making GUCY2C Receptor Ligands

A variety of methods of making polypeptides are known in the art and can be used to make GUCY2C polypeptide ligands. For example, GUCY2C polypeptide ligands can be recombinantly produced by utilizing a host cell system engineered to express a nucleic acid encoding a GUCY2C polypeptide ligand. Where a GUCY2C polypeptide ligand is recombinantly produced, any expression system can be used. Known expression systems include, without limitation, for example, egg, baculovirus, plant, yeast, or mammalian cells. Alternatively or additionally, a GUCY2C polypeptide ligand can be purified from natural sources.

Alternatively or additionally, a GUCY2C polypeptide ligand can be partially or fully prepared by chemical synthesis, including standard solid phase (or solution phase) peptide synthesis methods, as is known in the art. In addition, the nucleic acids encoding these polypeptides may be synthesized using commercially available oligonucleotide synthesis instrumentation and the proteins may be produced recombinantly using standard recombinant production systems.

Other suitable GUCY2C polypeptide ligands include peptide mimetics that mimic the three-dimensional structure of a GUCY2C polypeptide ligand. Such peptide mimetics may have significant advantages over naturally occurring peptides including, for example, more economical production, greater chemical stability, enhanced pharmacological properties (half-life, absorption, potency, efficacy, etc), altered specificity (e.g., broad-spectrum biological activities, reduced antigenicity and others). In some embodiments, mimetics are molecules that mimic elements of GUCY2C polypeptide ligand secondary structure. Peptide backbones of proteins exist mainly to orient amino acid side chains in such a way as to facilitate molecular interactions, such as those of a receptor and ligand. A peptide mimetic is expected to permit molecular interactions similar to the natural molecule. Peptide analogs are commonly used in the pharmaceutical industry as non-peptide drugs with properties analogous to those of the template peptide. These types of compounds are also referred to as peptide mimetics or peptidomimetics (see, e.g., Fauchere, Adv. Drug Res., 1986, 15: 29-69; Veber & Freidinger, 1985, Trends Neurosci., 1985, 8: 392-396; Evans et al., J. Med. Chem., 1987, 30: 1229-1239) and are usually developed with the aid of computerized molecular modeling.

Generally, peptide mimetics are structurally similar to a paradigm polypeptide (i.e., a polypeptide that has a biochemical property or pharmacological activity), but have one or more peptide linkages optionally replaced by a non-peptide linkage. The use of peptide mimetics can be enhanced through the use of combinatorial chemistry to create drug libraries. The design of peptide mimetics can be aided by identifying amino acid mutations that increase or decrease the binding of a peptide to, for example, a GUCY2C receptor. Approaches that can be used include the yeast two hybrid method (see, for example, Chien et al., Proc. Natl. Acad. Sci. USA, 1991, 88: 9578-9582) and using the phase display method. The two-hybrid method detects protein-protein interactions in yeast (Field et al., Nature, 1989, 340: 245-246). The phage display method detects the interaction between an immobilized protein and a protein that is expressed on the surface of phages such as lambda and M 13 (Amberg et al., Strategies, 1993, 6: 2-4; Hogrefe et al., Gene, 1993, 128: 119-126). These methods allow positive and negative selection of peptide-protein interactions and the identification of the sequences that determine these interactions.

In some peptide synthesis methods, an amino group of one amino acid (or amino acid derivative) is linked to a carboxyl group of another amino acid (or amino acid derivative) that has been activated by reacting it with a reagent such as dicyclohexylcarbodiimide (DCC). When the free amino group attacks the activated carboxyl group, a peptide bond is formed and dicyclohexylurea is released. In such methods, other potentially reactive groups (such as the α-amino group of the N-terminal amino acid or amino acid derivative and the carboxyl group of the C-terminal amino acid or amino acid derivative) may be blocked (“protected”) from participating in the chemical reaction. Thus, only particular active groups react such that the desired product is formed. Blocking groups useful for this purpose include without limitation tertbutoxycarbonyl groups (t-Boc) and benzoyloxycarbonyl groups to protect amine groups; and simple esters (such as methyl and ethyl groups) and benzyl esters to protect carboxyl groups. Blocking groups can typically be subsequently removed with a treatment that leaves peptide bonds intact (for example, treatment with dilute acid). This process of protecting reacting groups that should not react, coupling to form a peptide bond, and deprotecting reactive groups may be repeated. A peptide may be synthesized by sequentially adding amino acids to a growing peptide chain.

Both liquid-phase and solid phase peptide synthesis methods are suitable for use in accordance with the present disclosure. In solid-phase peptide synthesis methods, the growing peptide chain is typically linked to an insoluble matrix (such as, for example, polystyrene beads) by linking the carboxyl terminal amino acid to the matrix. At the end of synthesis, the peptide can be released from the matrix using a cleaving reagent that does not disrupt peptide bonds, such as hydrofluoric acid (HF). Protecting groups are also typically removed at this time. Automated, high throughput, and/or parallel peptide synthesis methods may also be used in accordance with the present disclosure. For more information about peptide synthesis methods, see, e.g., Merrifield (1969) Adv Enzymol Relat Areas Mol. Biol., 32:221-96; Fridkin et al., (1974) Annu Rev Biochem., 43 (0):419-43; Merrifield (1997) Methods in Enzymology, 289:3-13; Sabatino et al., (2009) Curr Opin Drug Discov Devel., 11(6):762-70.

### Detectable Moieties

In some embodiments, GUCY2C ligands described herein are conjugated to a detectable moiety. A detectable moiety may be any entity that allows detection of a GUCY2C ligand after binding to a tissue or localization at a system of interest. Any of a wide variety of detectable agents can be used as detectable moieties (e.g., labeling moieties). A detectable moiety may be directly detectable or indirectly detectable. Examples of detectable moieties include, but are not limited to, various ligands, radionuclides (e.g., ³H, ¹⁴C, ¹⁸F, ¹⁹F, ³²P, ³⁵S, ¹³⁵I, ¹²⁵I, ¹²⁴I, ¹²³I, ⁶⁴Cu, ¹⁸⁷Re, ¹¹¹In, ⁹⁰Y, ^{99m}Tc, ¹⁷⁷Lu, etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.), nanoclusters, paramagnetic metal ions, enzymes, colorimetric labels (such as, for example, dyes, colloidal gold, and the like), biotin, dioxigenin, haptens, and proteins for which antisera or monoclonal antibodies are available.

In certain embodiments, a detectable moiety comprises a fluorescent label. Numerous known fluorescent labeling moieties of a wide variety of chemical structures and physical characteristics are suitable for use in the practice of methods of diagnosis of the present disclosure. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, β carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, Spectrum Red™, Spectrum Green™, cyanine dyes (e.g., Cy-3™, Cy-5™, Cy-3.5™, Cy-5.5™ etc.), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660, Alexa Fluor 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable fluorescent dyes and methods for coupling fluorescent dyes to other chemical entities such as proteins and peptides are described in, e.g., “The Handbook of Fluorescent Probes and Research Products”, 9^{th} Ed., Molecular Probes, Inc., Eugene, Oreg.

Favorable properties of fluorescent labeling agents include high molar absorption coefficient, high fluorescence quantum yield, and photostability. In certain embodiments, labeling fluorophores desirably exhibit absorption and emission wavelengths in the visible (i.e., between 400 and 750 nm) rather than in the ultraviolet range of the spectrum (i.e., lower than 400 nm).

In certain embodiments, a detectable moiety comprises an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a GUCY2C ligand using a linker group such as a carbodiimide, a diisocyanate, a glutaraldehyde, and the like.

In certain embodiments, a detectable moiety comprises a radioisotope that is detectable by Single Photon Emission Computed Tomography (SPECT) or Position Emission Tomography (PET). Examples of such radionuclides include, but are not limited to, iodine-131 (¹³¹I), iodine-124 (¹²⁴I), iodine-125 (¹²⁵I), bismuth-212 (²¹²Bi), bismuth-213 (²¹³Bi), astatine-221 (²¹¹At), copper-67 (⁶⁷Cu), copper-64 (⁶⁴Cu), rhenium-186 (¹⁸⁶Re), rhenium-186 (¹⁸⁸Re), phosphorus-32 (³²P), samarium-153 (¹⁵³Sm), lutetium-177 (¹¹⁷Lu), technetium-99m (^{99m}Tc), gallium-67 (⁶⁷Ga), indium-111 (¹¹¹In), and thallium-201 (²⁰¹Tl).

In certain embodiments, a detectable moiety comprises a radioisotope that is detectable by Gamma camera. Examples of such radioisotopes include, but are not limited to, iodine-131 (¹³¹I) and technetium-99m (^{99m}Tc).

In certain embodiments, a detectable moiety comprises a paramagnetic metal ion that is a good contrast enhancer in Magnetic Resonance Imaging (MRI). Examples of such paramagnetic metal ions include, but are not limited to, gadolinium III (Gd³⁺), chromium III (Cr³⁺), dysprosium III (Dy³⁺), iron III (Fe³⁺), manganese II (Mn²⁺), and ytterbium III (Yb³⁺). In certain embodiments, the detection entity comprises gadolinium III (Gd³⁺). Gadolinium is an FDA-approved contrast agent for MRI, which accumulates in abnormal tissues causing these abnormal areas to become very bright (enhanced) on the magnetic resonance image. Gadolinium is known to provide great contrast between normal and abnormal tissues in different areas of the body, in particular in the brain.

In certain embodiments, a detectable moiety comprises a stable paramagnetic isotope detectable by nuclear magnetic resonance spectroscopy (MRS). Examples of suitable stable paramagnetic isotopes include, but are not limited to, carbon-13 (¹³C) and fluorine-19 (¹⁹F).

### Therapeutic Moieties

In some embodiments, a GUCY2C ligand described herein is conjugated to a therapeutic moiety (e.g., a neuroprotective agent). Nonlimiting exemplary neuroprotective agents include L-dopa, dopamine agonists (e.g., apomorphine, bromocriptine, pergolide, ropinirole, pramipexole, or cabergoline), adenosine A2a antagonists (Shah et al., Curr. Opin. Drug Discov. Devel. 13:466-80 (2010)); serotonin receptor agonists; continuous-release levodopa (Sinemet CR®, MSD, Israel); continuous duodenal levodopa administration (Duodopa®, Abbott, UK); catechol-O-methyltransferase (COMT) inhibitors (e.g., Stalevo®, Novartis Pharma, USA; entacapone (Comtan®, Novartis Pharma, USA)); tolcapone; coenzyme Q10, and/or MAO-B inhibitors (e.g., Selegiline or Rasagiline). Additional neuroprotective agents are described in, e.g., Hart et al., Mov. Disord. 24: 647-54 (2009).

In certain embodiments, GUCY2C ligand conjugate of the present disclosure is used in directed enzyme prodrug therapy. In a directed enzyme prodrug therapy approach, a directed/targeted enzyme and a prodrug are administered to a subject, wherein the targeted enzyme is specifically localized to a portion of the subject's body where it converts the prodrug into an active drug. The prodrug can be converted to an active drug in one step (by the targeted enzyme) or in more than one step. For example, the prodrug can be converted to a precursor of an active drug by the targeted enzyme. The precursor can then be converted into the active drug by, for example, the catalytic activity of one or more additional targeted enzymes, one or more non-targeted enzymes administered to the subject, one or more enzymes naturally present in the subject or at the target site in the subject (e.g., a protease, phosphatase, kinase or polymerase), by an agent that is administered to the subject, and/or by a chemical process that is not enzymatically catalyzed (e.g., oxidation, hydrolysis, isomerization, epimerization, etc.).

### Methods of Conjugation

In some embodiments of the present disclosure, a GUCY2C receptor ligand is conjugated to a payload moiety, e.g., a detectable moiety and/or a therapeutic moiety. Conjugation is not limited to particular modes of conjugation. For example, two entities may be covalently conjugated directly to each other. Alternatively, two entities may be indirectly conjugated to each other, such as via a linker entity.

In some embodiments, a GUCY2C receptor ligand is directly covalently linked to a payload moiety. Such direct covalent conjugation can be through a linkage (e.g., a linker or linking entity) such as an amide, ester, carbon-carbon, disulfide, carbamate, ether, thioether, urea, amine, or carbonate linkage. Covalent conjugation can be achieved by taking advantage of functional groups present on the GUCY2C ligand and/or payload moiety. Alternatively or additionally, a non-critical amino acid may be replaced by another amino acid that will introduce a useful group (such as amino, carboxy or sulfhydryl) for coupling purposes. Alternatively or additionally, an additional amino acid may be added to a GUCY2C ligand and/or payload moiety to introduce a useful group (such as amino, carboxy or sulfhydryl) for coupling purposes. Suitable functional groups that can be used to attach moieties together include, but are not limited to, amines, anhydrides, hydroxyl groups, carboxy groups, thiols, and the like. An activating agent, such as a carbodiimide, can be used to form a direct linkage. A wide variety of activating agents are known in the art and are suitable for conjugating one entity to a second entity.

In some embodiments, a GUCY2C receptor ligand is indirectly covalently linked to a payload moiety via a linker group. Such a linker group may also be referred to as a linker or a linking entity. This can be accomplished by using any number of stable bifunctional agents well known in the art, including homofunctional and heterofunctional agents (for examples of such agents, see, e.g., Pierce Catalog and Handbook). The use of a bifunctional linker differs from the use of an activating agent in that the former results in a linking moiety being present in the resulting conjugate (agent), whereas the latter results in a direct coupling between the two moieties involved in the reaction. The role of a bifunctional linker may be to allow reaction between two otherwise inert moieties. Alternatively or additionally, the bifunctional linker that becomes part of the reaction product may be selected such that it confers some degree of conformational flexibility to the GUCY2C ligand (e.g., the bifunctional linker comprises a straight alkyl chain containing several atoms, for example, the straight alkyl chain contains between 2 and 10 carbon atoms). Alternatively or additionally, the bifunctional linker may be selected such that the linkage formed between a GUCY2C ligand and a payload moiety is cleavable, e.g., hydrolyzable (for examples of such linkers, see e.g. U.S. Pat. Nos. 5,773,001; 5,739,116 and 5,877,296). Such linkers, for example, may be used when higher activity of certain entities, such as a therapeutic moiety, is observed after hydrolysis of the conjugate. Exemplary mechanisms by which a payload moiety may be cleaved from a GUCY2C ligand include hydrolysis in the acidic pH of the lysosomes (hydrazones, acetals, and cis-aconitate-like amides), peptide cleavage by lysosomal enzymes (the capthepsins and other lysosomal enzymes), and reduction of disulfides). Another mechanism by which such an entity is cleaved from a GUCY2C ligand includes hydrolysis at physiological pH extra- or intra-cellularly. This mechanism is useful when the crosslinker used to couple one entity to another entity is a biodegradable/bioerodible component, such as polydextran and the like.

For example, hydrazone-containing GUCY2C ligand conjugates can be made with introduced carbonyl groups that provide the desired release properties. GUCY2C ligand conjugates can also be made with a linker that comprises an alkyl chain with a disulfide group at one end and a hydrazine derivative at the other end. Linkers containing functional groups other than hydrazones also have the potential to be cleaved in the acidic environment of lysosomes. For example, GUCY2C ligand conjugates can be made from thiol-reactive linkers that contain a group other than a hydrazone that is cleavable intracellularly, such as esters, amides, and acetals/ketals.

Another example of class of pH sensitive linkers are the cis-aconitates, which have a carboxylic acid group juxtaposed to an amide group. The carboxylic acid accelerates amide hydrolysis in the acidic lysosomes. Linkers that achieve a similar type of hydrolysis rate acceleration with several other types of structures can also be used.

Another potential release method for GUCY2C ligand conjugates is the enzymatic hydrolysis of peptides by the lysosomal enzymes. In one example, a GUCY2C ligand is attached via an amide bond to para-aminobenzyl alcohol and then a carbamate or carbonate is made between the benzyl alcohol and a payload moiety. Cleavage of the peptide leads to collapse of the amino benzyl carbamate or carbonate, and release of the payload moiety. In another example, a phenol can be cleaved by collapse of the linker instead of the carbamate. In another variation, disulfide reduction is used to initiate the collapse of a para-mercaptobenzyl carbamate or carbonate.

Useful linkers that can be used as a linking entity include, without limitation, polyethylene glycol, a copolymer of ethylene glycol, a polypropylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid, a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group.

The present disclosure also encompasses GUCY2C ligand conjugates that involve non-covalent association. Examples of non-covalent interactions include, but are not limited to, hydrophobic interactions, electrostatic interactions, dipole interactions, van der Waals interactions, and hydrogen bonding. Irrespective of the nature of the binding, interaction, or coupling, the association between a GUCY2C ligand and a payload moiety is, in some embodiments, selective, specific and strong enough so that the payload moiety does not dissociate from the GUCY2C ligand before or during transport/delivery to and into a target.

### Administration

GUCY2C ligand conjugates described herein can be used to diagnose and/or treat PD, e.g., subjects suffering from or susceptible to PD. The route and/or mode of administration of a GUCY2C ligand conjugate described herein can vary depending upon the desired results. One with skill in the art, i.e., a physician, is aware that dosage regimens can be adjusted to provide the desired response, e.g., a detectable response for diagnosis and/or a therapeutic response.

Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. The mode of administration is left to the discretion of the practitioner.

In some instances, a GUCY2C ligand conjugate described herein can effectively cross the blood brain barrier (BBB) and enter the brain. Alternatively or additionally, a GUCY2C ligand conjugate can be delivered using techniques designed to permit or to enhance the ability of the formulation to cross the BBB. Such techniques are known in the art (e.g., WO 89/10134; Cloughesy et al., J. Neurooncol. 26:125-132 (1995); and Begley, J. Pharm. Pharmacol. 48:136-146 (1996)). Components of a formulation can also be modified (e.g., chemically) using methods known in the art to facilitate their entry into the CNS.

For example, physical methods of transporting compositions across the BBB include, but are not limited to, circumventing the BBB entirely, or by creating openings in the BBB. Circumvention methods include, but are not limited to, direct injection into the brain (see e.g., Papanastassiou et al., Gene Therapy 9: 398-406 (2002)) and implanting a delivery device in the brain (see e.g., Gill et al., Nature Med. 9: 589-595 (2003); and Gliadel Wafers™, Guildford Pharmaceutical). Methods of creating openings in the barrier include, but are not limited to, ultrasound (see e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), and permeabilization by, e.g., bradykinin or permeabilizer A-7 (see, e.g., U.S. Pat. Nos. 5,112,596, 5,268,164, 5,506,206, and 5,686,416).

Lipid-based methods can also be used to transport a GUCY2C ligand conjugate across the BBB. Exemplary, nonlimiting methods include encapsulating a GUCY2C ligand conjugate in liposomes that are coupled to a targeting agent (e.g., an antibody that binds to receptors on vascular endothelium of the BBB (see, e.g., U.S. Patent Publ. No. 20020025313)). In certain other embodiments, a targeting agent is coated in low-density lipoprotein particles (see, e.g., U.S. Patent Publ. No. 20040204354) or apolipoprotein E (see, e.g., U.S. Patent Publ. No. 20040131692).

In some embodiments, the ability of a GUCY2C ligand conjugate to cross the BBB is measured using assays, e.g., using in vitro models of BBB, known in the art (see, e.g., Mensch et al., Eur. J. Pharm. Biopharm. 74:495-502 (2010); Cecchelli et al., Nature Rev. Drug Discov. 6:650-661 (2007)). Nonlimiting, exemplary assay systems include, e.g., endothelial cells from different species, placed in cocultures mostly with rat astrocytes (Hayashi et al., Glia 19:13-26 (1997); Parran et al., Neurotoxicology 26:77-88 (2005)) or placed in monolayers of endothelial cells induced to present BBB characteristics (Perriere et al., Brain Res. 1150:1-13 (2007); Roux et al., Cell. Mol. Neurobiol. 25:41-58 (2005); van Bree et al., Pharm. Res. 5:369-371 (1988)); human brain vascular endothelial cell monolayer models (Cucullo et al., Epilepsia 48:505-516 (2007); Ishihara et al., J. Neuropathol. Exp. Neurol. 67:435-448 (2008)); bovine brain microvascular endothelial cells (BMEC); bovine brain endothelial cell (BBEC) culture; and rat brain microvascular endothelial cells (RBE4) (Balbuena et al., Toxicol. Sci. 114:260-71 (2010)). Some assay systems include mixed cells of bovine, rat, and porcine origin (Roux et al., Cell. Mol. Neurobiol. 25:41-58 (2005)); or BBB systems with cells from the same animal species (Nakagawa et al., Neurochem. Int. 54:253-263 (2009); Perriere et al., Brain Res. 1150:1-13 (2007)); Another exemplary assay system is a parallel artificial membrane permeability assay (PAMPA) (Mensch et al., Eur. J. Pharm. Biopharm. 74:495-502 (2010)).

In some embodiments, at least about 10%, 15%, 20%, 25%, 30%, 35%, 40%, 45%, 50%, 55%, 60%, 65%, 70%, 75%, 80%, 85%, 90%, 95%, or 100%, of a GUCY2C ligand conjugate crosses the BBB in one or more of such in vitro assay systems. In some embodiments, at least about 10%, 15%, 20%, 25%, 30%, 35%, 40%, 45%, 50%, 55%, 60%, 65%, 70%, 75%, 80%, 85%, 90%, 95%, or 100%, of a GUCY2C ligand conjugate crosses the BBB in vivo upon administration to a subject.

In some embodiments, a GUCY2C ligand conjugate is delivered to the CNS of a subject, e.g., by administering into the cerebrospinal fluid (CSF) of a subject, e.g., in need of treatment. As used herein, intrathecal administration (also referred to as intrathecal injection) refers to an injection into the spinal canal (intrathecal space surrounding the spinal cord). Various techniques may be used including, without limitation, lateral cerebroventricular injection through a burrhole or cisternal or lumbar puncture or the like. Exemplary methods are described in Lazorthes et al., Adv. Tech. Stand. Neurosurg. 18:143-192 (1991), and Omaya, Cancer Drug Deliv. 1:169-179 (1984).

In some instances, a GUCY2C ligand conjugate described herein is administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers. In some situations, a GUCY2C ligand conjugate described herein is introduced into the central nervous system, circulatory system or gastrointestinal tract by any suitable route, including intraventricular injection, intrathecal injection, paraspinal injection, epidural injection, enema, and by injection adjacent to a peripheral nerve.

Specifically, various devices can be used for intrathecal delivery of a GUCY2C ligand conjugates described herein. In some embodiments, a device for intrathecal administration contains a fluid access port (e.g., injectable port); a hollow body (e.g., catheter) having a first flow orifice in fluid communication with the fluid access port and a second flow orifice configured for insertion into spinal cord; and a securing mechanism for securing the insertion of the hollow body in the spinal cord. Various other devices may be used to effect intrathecal administration of a therapeutic composition. For example, formulations containing a GUCY2C ligand conjugate can be administered using an Ommaya reservoir that is in common use for intrathecally administering drugs for meningeal carcinomatosis (Lancet 2: 983-84, 1963). More specifically, in this method, a ventricular tube is inserted through a hole formed in the anterior horn and is connected to an Ommaya reservoir installed under the scalp, and the reservoir is subcutaneously punctured to intrathecally deliver a GUCY2C ligand conjugate, which is injected into the reservoir. Other devices for intrathecal administration of therapeutic compositions or formulations to an individual are described in U.S. Pat. No. 6,217,552. Alternatively or additionally, a GUCY2C ligand conjugate can be intrathecally given, for example, by a single injection, or continuous infusion. It should be understood that the dosage treatment may be in the form of a single dose administration or multiple doses.

In some embodiments, intrathecal administration can be performed by either lumbar puncture (i.e., slow bolus) or via a port-catheter delivery system (i.e., infusion or bolus).

Relative to intravenous administration, a single dose volume suitable for intrathecal administration is typically small. Typically, intrathecal delivery maintains the balance of the composition of the CSF as well as the intracranial pressure of the subject. In some embodiments, intrathecal delivery is performed absent the corresponding removal of CSF from a subject. In some embodiments, a suitable single dose volume may be e.g., less than about 10 ml, 8 ml, 6 ml, 5 ml, 4 ml, 3 ml, 2 ml, 1.5 ml, 1 ml, or 0.5 ml. In some embodiments, a suitable single dose volume may be about 0.5-5 ml, 0.5-4 ml, 0.5-3 ml, 0.5-2 ml, 0.5-1 ml, 1-3 ml, 1-5 ml, 1.5-3 ml, 1-4 ml, or 0.5-1.5 ml. In some embodiments, intrathecal delivery according to the present invention involves a step of removing a desired amount of CSF first. In some embodiments, less than about 10 ml (e.g., less than about 9 ml, 8 ml, 7 ml, 6 ml, 5 ml, 4 ml, 3 ml, 2 ml, 1 ml) of CSF is first removed before intrathecal administration. In those cases, a suitable single dose volume may be e.g., more than about 3 ml, 4 ml, 5 ml, 6 ml, 7 ml, 8 ml, 9 ml, 10 ml, 15 ml, or 20 ml.

Pulmonary administration can also be employed, e.g., by use of an inhaler or nebulizer, and formulation with an aerosolizing agent, or via perfusion in a fluorocarbon or synthetic pulmonary surfactant.

A GUCY2C ligand conjugate described herein can be formulated as a diagnostic and/or pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one situation, the physiologically acceptable excipients are sterile when administered to an animal. The physiologically acceptable excipient should be stable under the conditions of manufacture and storage and should be preserved against the contaminating action of microorganisms. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The diagnostic and/or pharmaceutical compositions, if desired, can also contain minor amounts of wetting or emulsifying agents, or pH buffering agents.

Liquid carriers can be used in preparing solutions, suspensions, emulsions, syrups, and elixirs. A GUCY2C ligand conjugate described herein can be suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

In other instances, a GUCY2C ligand conjugate described herein is formulated for intravenous administration. Compositions for intravenous administration can comprise a sterile isotonic aqueous buffer. The compositions can also include a solubilizing agent. Compositions for intravenous administration can optionally include a local anesthetic such as lidocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a GUCY2C ligand conjugate described herein is administered by infusion, it can be dispensed, for example, with an infusion bottle containing sterile pharmaceutical grade water or saline. Where a GUCY2C ligand conjugate described herein is administered by injection, an ampule of sterile water for injection or saline can be provided so that the ingredients can be mixed prior to administration.

A GUCY2C ligand conjugate described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a GUCY2C ligand conjugate described herein that is effective for diagnosing and/or treating PD can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner.

Compositions described herein (e.g., therapeutically effective amounts of compositions described herein) can be administered as single administrations or as multiple administrations. Such compositions can be administered at regular intervals, depending on the nature, severity and extent of the subject's condition (e.g., PD). In some embodiments, a therapeutically effective amount of a therapeutic agent (e.g., a GUCY2C ligand conjugate) is administered intrathecally periodically at regular intervals (e.g., once every year, once every six months, once every five months, once every three months, bimonthly (once every two months), monthly (once every month), biweekly (once every two weeks), or weekly).

As used herein, the term “therapeutically effective amount” is largely determined based on the total amount of a diagnostic or therapeutic agent contained in pharmaceutical compositions described herein. Generally, a therapeutically effective amount is sufficient to achieve a meaningful benefit to a subject (e.g., diagnosing, treating, modulating, curing, preventing and/or ameliorating PD). For example, a therapeutically effective amount can be an amount sufficient to achieve a desired therapeutic and/or prophylactic effect, such as an amount sufficient to treat PD or the symptoms thereof. Generally, the amount of a therapeutic agent (e.g., a GUCY2C ligand conjugate) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays can optionally be employed to identify optimal dosage ranges. A therapeutically effective amount can be administered in a dosing regimen that can include multiple unit doses.

In some embodiments, a therapeutically effective dose ranges from about 0.005 mg/kg brain weight to 500 mg/kg brain weight, e.g., from about 0.005 mg/kg brain weight to 400 mg/kg brain weight, from about 0.005 mg/kg brain weight to 300 mg/kg brain weight, from about 0.005 mg/kg brain weight to 200 mg/kg brain weight, from about 0.005 mg/kg brain weight to 100 mg/kg brain weight, from about 0.005 mg/kg brain weight to 90 mg/kg brain weight, from about 0.005 mg/kg brain weight to 80 mg/kg brain weight, from about 0.005 mg/kg brain weight to 70 mg/kg brain weight, from about 0.005 mg/kg brain weight to 60 mg/kg brain weight, from about 0.005 mg/kg brain weight to 50 mg/kg brain weight, from about 0.005 mg/kg brain weight to 40 mg/kg brain weight, from about 0.005 mg/kg brain weight to 30 mg/kg brain weight, from about 0.005 mg/kg brain weight to 25 mg/kg brain weight, from about 0.005 mg/kg brain weight to 20 mg/kg brain weight, from about 0.005 mg/kg brain weight to 15 mg/kg brain weight, from about 0.005 mg/kg brain weight to 10 mg/kg brain weight.

In some embodiments, a therapeutically effective dose is greater than about 0.1 mg/kg brain weight, greater than about 0.5 mg/kg brain weight, greater than about 1.0 mg/kg brain weight, greater than about 3 mg/kg brain weight, greater than about 5 mg/kg brain weight, greater than about 10 mg/kg brain weight, greater than about 15 mg/kg brain weight, greater than about 20 mg/kg brain weight, greater than about 30 mg/kg brain weight, greater than about 40 mg/kg brain weight, greater than about 50 mg/kg brain weight, greater than about 60 mg/kg brain weight, greater than about 70 mg/kg brain weight, greater than about 80 mg/kg brain weight, greater than about 90 mg/kg brain weight, greater than about 100 mg/kg brain weight, greater than about 150 mg/kg brain weight, greater than about 200 mg/kg brain weight, greater than about 250 mg/kg brain weight, greater than about 300 mg/kg brain weight, greater than about 350 mg/kg brain weight, greater than about 400 mg/kg brain weight, greater than about 450 mg/kg brain weight, greater than about 500 mg/kg brain weight.

In some embodiments, a therapeutically effective dose can be expressed as mg/kg body weight. As one skilled in the art would appreciate, brain weights and body weights can be correlated (see, e.g., Dekaban, Ann. Neurol. 4:345-56 (1978)).

In some embodiments, a therapeutically effective dose can be expressed as mg/15 cc of CSF. As one skilled in the art would appreciate, therapeutically effective doses based on brain weights and body weights can be converted to mg/15 cc of CSF. For example, the volume of CSF in adult humans is approximately 150 mL (Johanson et al., Cerebrospinal Fluid Res. 14:5:10 (2008)). Therefore, single dose injections of 0.1 mg to 50 mg protein to adults would be approximately 0.01 mg/15 cc of CSF (0.1 mg) to 5.0 mg/15 cc of CSF (50 mg) doses in adults.

It is to be further understood that for any particular subject, specific dosage regimens can be adjusted over time according to the individual need and the professional judgment of the person administering or supervising the administration of a GUCY2C ligand conjugate and that dosage ranges set forth herein are exemplary only and are not intended to limit the scope or practice of the claimed invention.

In some instances, a diagnostic or pharmaceutical composition described herein is in unit dosage form, e.g., as a tablet, capsule, powder, solution, suspension, emulsion, granule, or suppository. In such form, a diagnostic or pharmaceutical composition can be sub-divided into unit doses containing appropriate quantities of a GUCY2C ligand conjugate described herein. The unit dosage form can be a packaged diagnostic or pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

### Kits

A GUCY2C ligand conjugate described herein (e.g., a pharmaceutical or diagnostic composition comprising a GUCY2C ligand conjugate) can be provided in a kit. In some instances, the kit includes (a) a container that contains a GUCY2C ligand conjugate described herein (e.g., a pharmaceutical or diagnostic composition comprising a GUCY2C ligand conjugate) and, optionally (b) informational material. The informational material can be descriptive, instructional, marketing or other material that relates to the methods described herein and/or the use of a GUCY2C ligand conjugate, e.g., for diagnostic or therapeutic benefit.

The informational material of the kits is not limited in its form. In some instances, the informational material can include information about production of a GUCY2C ligand conjugate, molecular weight of a GUCY2C ligand conjugate, concentration, date of expiration, batch or production site information, and so forth. In other situations, the informational material relates to methods of administering a GUCY2C ligand conjugate, e.g., in a suitable amount, manner, or mode of administration (e.g., a dose, dosage form, or mode of administration described herein). The method can be a method of diagnosing a subject having or at risk of PD, and/or treating a subject having PD.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about a GUCY2C ligand conjugate therein and/or their use in the methods described herein. The informational material can also be provided in any combination of formats.

In addition to a GUCY2C ligand conjugate, the kit can include other ingredients, such as a solvent or buffer, a stabilizer, or a preservative. The kit can also include other agents, e.g., a second or third agent, e.g., other diagnostic or therapeutic agents. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for a GUCY2C ligand conjugate or other agents. In some cases, the kit contains separate containers, dividers or compartments for a GUCY2C ligand conjugate and informational material. For example, a GUCY2C ligand conjugate can be contained in a bottle, vial, or syringe, and the informational material can be contained in a plastic sleeve or packet. In other situations, the separate elements of the kit are contained within a single, undivided container. For example, a GUCY2C ligand conjugate can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of a GUCY2C ligand conjugate. The containers can include a unit dosage, e.g., a unit that includes a GUCY2C ligand conjugate. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of a GUCY2C ligand conjugate, e.g., a syringe or other suitable delivery device. The device can be provided preloaded with a GUCY2C ligand conjugate, e.g., in a unit dose, or can be empty, but suitable for loading.

### Diagnosis of Parkinson's Disease

The present disclosure encompasses the discovery that GUCY2C receptor, e.g., on mDA neurons in the brain, e.g., in the substantia nigra, can be effectively targeted for diagnostic and/or therapeutic purposes. Current imaging technology used to distinguish a Parkinsonian brain from a healthy brain are based on three different measurements of presynaptic dopamine release into the caudate putamen (where mDA neurons project): (i) presynaptic dopamine transporters (DAT) that can be assessed with a variety of PET and SPECT tracers (i.e., ¹²³I-β-CIT, ¹²³I-FP-CIT, ¹²³I-altropane, and ¹¹C-CFT); (ii) ¹⁸F-dopa (fluorodopa) PET provides a marker of terminal dopa decarboxylase activity and dopamine turnover; (iii) vesicle monoamine transporter availability in dopamine terminals can be examined with either ¹¹C- or ¹⁸F-dihydrotetrabenazine PET (Brooks, J. Nucl. Med. 51:596-609 (2010)). However, these methods are not effective in identifying a presymptomatic Parkinsonian brain. GUCY2C ligand conjugates described herein can be used to measure the number of mDA neurons within a patient, e.g., a presymtomatic PD patient.

In certain embodiments, provided are methods comprising administering a composition comprising a GUCY2C ligand conjugate described herein to a subject having or suspected of having PD, such that the GUCY2C ligand conjugate binds specifically to a GUCY2C receptor (e.g., a GUCY2C receptor on an mDA neuron). In some embodiments, such methods are useful in diagnosis of PD.

In some embodiments, a GUCY2C ligand conjugated to a detectable moiety, e.g., a detectable moiety described herein, as administered to a subject having or suspected of having PD, and the level of detectable moiety bound to mDA neurons (e.g., in the substantia nigra) is measured, wherein a level of detectable moiety bound to mDA neurons than is less than a predetermined level (e.g., a level of detectable moiety bound to mDA neurons in a control subject) indicates the subject has or is at risk of developing PD.

In some embodiments, the detectable moiety is a radionuclide, such as iodine-124 (¹²⁴I), and the level of detectable moiety bound to mDA neurons is measured by Single Photon Emission Computed Tomography (SPECT) or Position Emission Tomography (PET).

### Treatment of Parkinson's Disease

The present disclosure encompasses the finding that GUCY2C ligands are useful, among other things, in targeting mDA neurons in the substantia nigra. Accordingly, in some embodiments, a GUCY2C ligand conjugate is provided to the central nervous system of a subject, e.g., a subject suffering from or susceptible to PD. In some embodiments, a GUCY2C ligand conjugate (e.g., including a therapeutic moiety) is provided to one or more of target cells or tissues of brain, spinal cord, and/or peripheral organs. In some embodiments, target cells or tissues include those cells or tissues that display a disease-associated pathology, symptom, or feature. In some embodiments, target cells or tissues include those cells or tissues in which GUCY2C receptor is expressed at a reduced level, compared to a reference. As used herein, a target tissue may be a brain target tissue, a spinal cord target tissue and/or a peripheral target tissue.

Compositions described herein can be provided directly into the CNS of a subject suffering from or at risk of developing PD, thereby achieving a therapeutic concentration within the affected cells and tissues of the CNS (e.g., the brain). For example, one or more a GUCY2C ligand conjugates can be provided to target cells or tissues of the brain, spinal cord and/or peripheral organs to PD. As used herein, the term “treat” or “treatment” refers to amelioration of one or more symptoms associated with the disease, prevention or delay of the onset of one or more symptoms of the disease, and/or lessening of the severity or frequency of one or more symptoms of the disease.

In some embodiments, treatment refers to partially or complete alleviation, amelioration, relief, inhibition, delaying onset, reducing severity and/or incidence of neurological impairment in a patient suffering from or susceptible to PD. As used herein, the term “neurological impairment” includes various symptoms associated with impairment of the central nervous system (e.g., the brain and spinal cord). Symptoms of neurological impairment may include, for example, bradykinesia; general slowing down of movements and of activities of daily living; lack of facial expression (hypomimia or masked facies); staring expression due to decreased frequency of blinking; impaired swallowing, which causes drooling; hypokinetic and hypophonic dysarthria; monotonous speech; small handwriting (micrographia); difficulties with repetitive and simultaneous movements; difficulty in arising from chair and turning over in bed; shuffling gait with short steps; decreased arm swing and other automatic movements; and start hesitation and freezing.

In some embodiments, treatment refers to decreased toxicity or loss of neuronal cells or tissues. In some embodiments, treatment refers to decreased loss of mDA neurons. In certain embodiments, loss of mDA neurons is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. In some embodiments, loss of mDA neurons is decreased by at least 1-fold, 2-fold, 3-fold, 4-fold, 5-fold, 6-fold, 7-fold, 8-fold, 9-fold or 10-fold as compared to a control.

In certain embodiments, treatment according to the present disclosure results in a reduction (e.g., about a 5%, 10%, 15%, 20%, 25%, 30%, 40%, 50%, 55%, 60%, 65%, 70%, 75%, 80%, 90%, 95%, 97.5%, 99% or more reduction) or a complete elimination of the presence, or alternatively the accumulation, of one or more pathological, clinical, or biological markers that are associated with PD. For example, in some embodiments, upon administration to a subject, a pharmaceutical composition described herein demonstrates or achieves a reduction in bradykinesia; general slowing down of movements and of activities of daily living; lack of facial expression (hypomimia or masked facies); staring expression due to decreased frequency of blinking; impaired swallowing, which causes drooling; hypokinetic and hypophonic dysarthria; monotonous speech; small handwriting (micrographia); difficulties with repetitive and simultaneous movements; difficulty in arising from chair and turning over in bed; shuffling gait with short steps; decreased arm swing and other automatic movements; and start hesitation and freezing.

In some embodiments, treatment refers to increased survival (e.g., survival time). For example, treatment can result in an increased life expectancy of a patient. In some embodiments, treatment results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with PD without treatment. In some embodiments, treatment results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with PD without treatment. In some embodiments, treatment results in long term survival of a patient. As used herein, the term “long term survival” refers to a survival time or life expectancy longer than about 40 years, 45 years, 50 years, 55 years, 60 years, or longer.

The term “improve,” “increase” or “reduce,” as used herein, indicates values that are relative to a control. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with PD, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having PD or having the potential to develop PD. In some instances, a subject to be treated is genetically predisposed to developing PD. For example, a subject to be treated has a mutation in a parkin gene or a DJ-1 gene.

In some embodiments, a subject is diagnosed as having or susceptible to PD according to a method of the present disclosure, and the subject is treated with a GUCY2C ligand conjugated to a therapeutic moiety and/or is treated with a known therapy for the treatment of PD.

In some embodiments, treatment of PD comprises administering to a subject in need thereof a GUCY2C receptor antagonist. In some embodiments, the antagonist is Rp-8-pCPT-cGMPS (see, e.g., Gong et al., Science 333:1642-6 (2011); Garin-Laflam et al., Am. J. Physiol. Gastrointest. Liver Physiol. 296:G740-G749 (2009)) or HS-142-1 (see, e.g., Ohyama et al., Life Sci. 52:PL153-7 (1993)).

In some embodiments, treatment of PD comprises administering to a subject in need thereof a binding agent that selectively binds to a GUCY2C ligand described herein (e.g., is a competitive binder of a GUCY2C ligand described herein). Methods of identifying and/or producing competitive binders of ligands are known in the art.

### Combination Therapy

In some embodiments, a GUCY2C ligand conjugate described herein is administered to a subject in combination with one or more additional therapies to treat PD or one or more symptoms of PD. For example, a GUCY2C ligand conjugate can be administered in combination with one or more of L-dopa, a dopamine agonist (e.g., apomorphine, bromocriptine, pergolide, ropinirole, pramipexole, or cabergoline), an adenosine A2a antagonist (Shah et al., Curr. Opin. Drug Discov. Devel. 13:466-80 (2010)); a serotonin receptor agonist; continuous-release levodopa (Sinemet CR®, MSD, Israel); continuous duodenal levodopa administration (Duodopa®, Abbott, UK); a catechol-β-methyltransferase (COMT) inhibitor (e.g., Stalevo®, Novartis Pharma, USA; entacapone (Comtan®, Novartis Pharma, USA)); tolcapone; a MAO-B inhibitor (e.g., Rasagiline); deep brain stimulation; or subthalamic nucleus-deep brain stimulation.

In some embodiments, combined administration of a GUCY2C ligand conjugate and a second agent results in an improvement in PD or a symptom thereof to an extent that is greater than one produced by either the a GUCY2C ligand conjugate or the second agent alone. The difference between the combined effect and the effect of each agent alone can be a statistically significant difference.

In some embodiments, combined administration of a GUCY2C ligand conjugate and a second agent allows administration of the second agent at a reduced dose, at a reduced number of doses, and/or at a reduced frequency of dosage compared to a standard dosing regimen approved for the second agent.

In some embodiments, an immunosuppressant agent known to the skilled artisan can be administered to a subject in combination with a GUCY2C ligand conjugate described herein. Exemplary immunosuppressant agents include, without limitation, cyclosporine, FK506, rapamycin, CTLA4-Ig, anti-TNF agents (such as etanercept), daclizumab (e.g., Zenapax™), anti-CD2 agents, anti-CD4 agents, and anti-CD40 agents.

### EXAMPLES

### Example 1

### Identification of GUCY2C in Midbrain Dopamine Neurons

GUCY2C is a known gut receptor that binds to heat stable enterotoxins produced by gut flora (resulting in diarrhea), and to guanylin and uroguanylin. This receptor, however, was discovered to be specific to midbrain dopamine (mDA) neurons of the adult brain using a 3 line BAC transgenic mouse embryonic stem cell (mESC) study.

### Methods

BAC Transgenic ESC Line Production.

RP23-236113 (GENSAT-modified) BAC for NURR1 and RP11-946K20 (Children's Hospital Oakland Research Institute; YFP recombineering into the entire coding sequence was performed as described in Tomishima et al., Stem Cells 25(1):39-45 (2007)) BAC for PITX3 were retrofitted with a Kan/G418 cassette for mammalian selection, and purified DNA was produced with the PSI Ψ Clone BAC DNA Kit according to the manufacturer's protocol and nucleo-fected into 6×10⁶ cells as described in Tomishima et al., Stem Cells 25(1):39-45 (2007). Nucleofected cells were plated onto Neo-resistant mouse embryonic fibroblasts (Globalstem) for 2 days, upon which G418 selection was performed at a concentration of 200 μg/ml for 7 to 10 days. Colonies were manually picked and expanded clonally and differentiated in 6-well plates as described in Barberi et al., Nat Biotechnol. 21(10):1200-1207 (2003). Reporter+ clones (observed at about day 10 for NURR1 and day 12 for PITX3 lines) were further characterized for proper protein expression in vitro using the rabbit anti-NURR1 antibody (Perseus Proteomics; 1:1,000) and rabbit anti-PITX3 antibody (Zymed; 1:100) to double stain with GFP (Abeam; 1:1,000). Upon confirmation of proper protein expression, reporter+ colonies were differentiated at large scale for 2 weeks, reporter+ and reporter− cells were FACS-purified, and RNA was isolated in TRIzol (Invitrogen) according to the manufacturer's protocol. qRT-PCR was then performed for each lines' corresponding RNA enrichment using the housekeeping gene HPRT to normalize the data transcripts. BAC FISH was performed with purified BAC DNA. The Nurr1::GFP;UbC::RFP and Hes5::GFP;UbC::RFP cell lines were clonally selected after viral transduction with a modified plasmid driving RFP under the ubiquitin C promoter (see Fasano et al., Cell Stem Cell. 1(1):87-99 (2007)).

mESC Culture, Differentiation, and FACS Preparation.

J1 mESCs were grown on mouse embryonic fibroblasts in mESC medium (knockout DMEM, 20% FBS, 2 mM glutamine, 0.1 mM MEM-nonessential amino acids, and 55 μM β-mercaptoethanol) supplemented with 1,400 U/ml LIF (ESGRO, Invitrogen). Differentiations for grafting and FACS were performed under a modified version of the MS5-based protocol previously reported (Barberi et al., Nat Biotechnol. 21(10):1200-1207 (2003)). Briefly, ESCs were trypsinized and plated onto a dense MS5 feeder layer at a concentration of 20,000 cells per 10-cm dish. The same protocol was applied, except using a different sonic hedgehog (SHH), C25II SHH(R&D Systems) at 50 ng/μl, and the whole protocol was shifted 1 day earlier (neural induction as day 4).

Initial sorting methods consisted of dissociation via resuspension in HBSS with 0.25% trypsin (1:100) for 1 hour at 37° C. and manual trituration and resuspension in N2 with propidium iodide (2 mg/ml). Propidium iodide-negative cells were plotted for PE and FITC, and cells were caught into FBS. Double sorting was performed by spinning down the reporter+-sorted cells and resuspending them in PBS+. All cells for grafting and in vitro analysis were resuspended in N2 with BDNF (20 ng/ml) and ascorbic acid (200 μM) at a concentration of between 50,000 and 100,000 cells per μl. For in vitro analysis, 1- to 2-μl drops were spread onto PO/Lam- or Matrigel-treated (according to the manufacturer's protocol) Permanox chamber slides (Labtek) for 5 minutes, resuspended in N2/BDNF/AA, and analyzed after 24 hours or 7 days.

Microarray Analysis.

mRNA transcriptome analyses were performed using Illumina Mouse-6 BeadChip technology for mRNA comparisons of 3 separate sorts for the Hes5::GFP^{+/-},Nurr1::GFP^{+/-}, and Pitx3::YFP^{+/-} populations. Data were analyzed using Partek software. Significance was based on an ANOVA analysis of all sorted populations, and significant gene lists were created under the criteria of an unadjusted P value of ≦0.05. Only transcripts enriched or depleted more than 2 fold were analyzed.

### Results

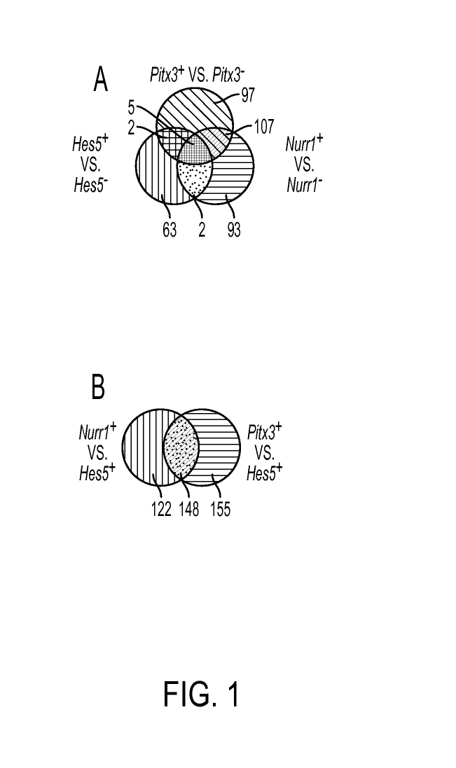

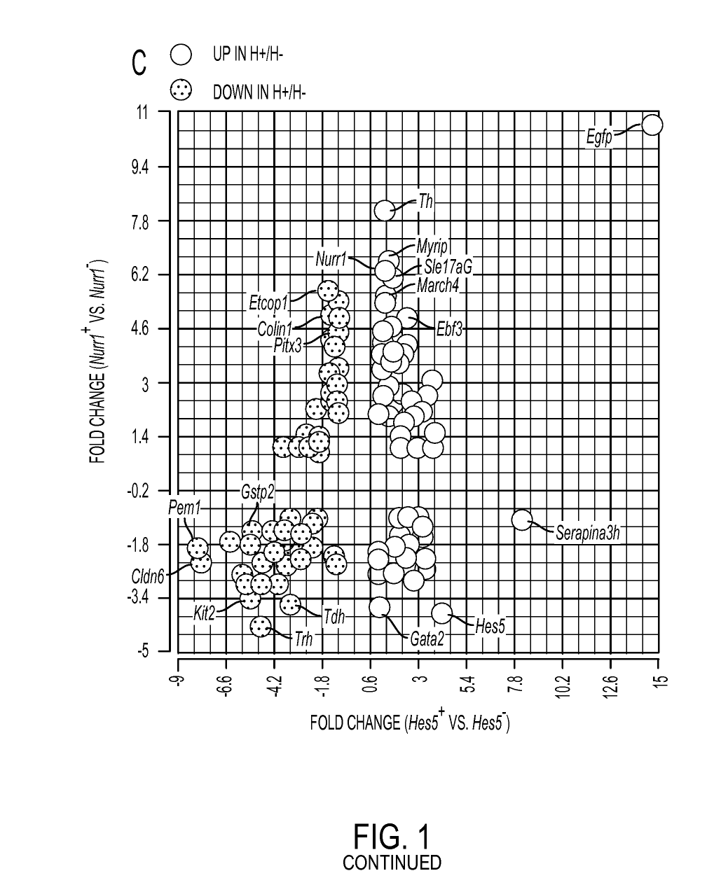

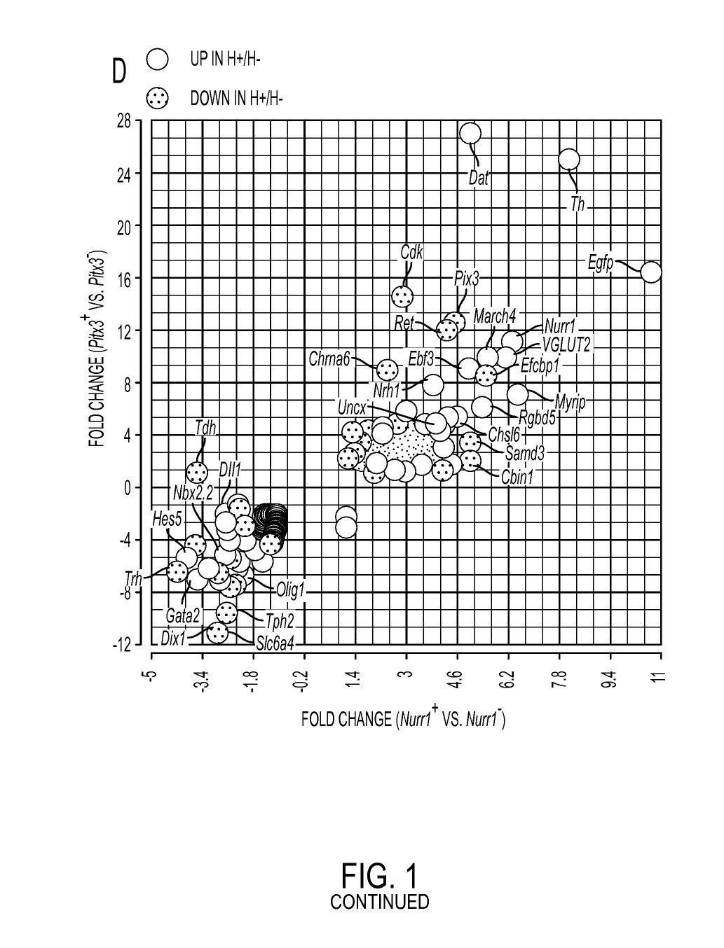

These 3 lines allowed for the FACS-purification of large amounts of neural subtypes under a standard mESC differentiation protocol into mDA neurons (Barberi et al., Nat. Biotechnol. 21:1200-1207 (2003)). Hes5::GFP+ (neural precursors), Nurr1::GFP+ (early midbrain dopamine), and Pitx3:: YFP+ (late midbrain dopamine) cells were each FACS-purified after two weeks of differentiation, and subjected to two paradigms of global transcriptome analysis: intraline (reporter+ versus reporter−) and interline analysis (only reporter+ cells between the three lines) (FIGS. 1A, 1B, respectively). Gene ontological analysis of transcripts that were integral to the cell membrane revealed 43 cell surface marker genes (see Table 1), of which the GUCY2C gene had the highest significance level (p-value=0.00011), and was enriched roughly 3 fold under both intraline and interline analysis. In addition, a human homologue of the nicotinic acetylcholine receptor gene CHRNA6 was identified (CHRNB3), which was specifically expressed by mDA neurons in vivo and in hESC-derived mDA neurons.

TABLE 1
Cell surface markers identified by gene ontology

| | p-value | Fold Change | p-value | Fold Change | p-value | Fold Change |
|---|---|---|---|---|---|---|
| Column ID | (N+P+)/H+ | (P+/N+)/H+ | (Nurr+ vs. Hes+) | (Nurr+ vs. Hes+) | (Pitx+ vs. Hes+) | (Pitx+ vs. Hes+) |
| RIT2 | 0.0023 | 9.72 | 0.0169 | 6.59 | 0.0021 | 14.33 |
| RET | 0.0002 | 8.30 | 0.0048 | 5.04 | 0.0001 | 13.66 |
| MARCH4 | 0.0004 | 6.68 | 0.0059 | 4.55 | 0.0003 | 9.81 |
| CHRNA6 | 0.0007 | 4.79 | 0.0402 | 2.50 | 0.0001 | 9.18 |
| CNTNAP4 | 0.0120 | 4.70 | 0.0451 | 3.87 | 0.0138 | 5.71 |
| NRN1 | 0.0012 | 4.57 | 0.0029 | 4.71 | 0.0037 | 4.44 |
| GLRA2 | 0.0165 | 4.52 | | | 0.0159 | 5.78 |
| SLC35O3 | 0.0003 | 4.40 | 0.0018 | 3.88 | 0.0005 | 4.98 |
| CBLN1 | 0.0010 | 4.26 | 0.0006 | 5.83 | 0.0124 | 3.11 |
| GPR158 | 0.0037 | 4.22 | 0.0288 | 3.16 | 0.0028 | 5.64 |
| CHL1 | 0.0041 | 4.09 | 0.0198 | 3.44 | 0.0049 | 4.86 |
| RGS9 | 0.0031 | 3.86 | 0.0175 | 3.20 | 0.0034 | 4.65 |
| CHST8 | 0.0005 | 3.79 | 0.0006 | 4.59 | 0.0045 | 3.12 |
| CACNG3 | 0.0497 | 3.73 | | | 0.0311 | 5.47 |
| KCNV2 | 0.0178 | 3.68 | 0.0142 | 4.82 | | |
| SCN33 | 0.0022 | 3.44 | 0.0140 | 2.87 | 0.0023 | 4.11 |
| ATP1B1 | 0.0023 | 3.12 | 0.0089 | 2.89 | 0.0039 | 3.37 |
| CHST1 | 0.0005 | 3.05 | 0.0165 | 2.13 | 0.0002 | 4.37 |
| GUCY2C | 0.0001 | 3.03 | 0.0015 | 2.54 | 0.0001 | 3.61 |
| SGPP2 | 0.0033 | 2.94 | 0.0119 | 2.75 | 0.0057 | 3.14 |
| D10BWG1379E | 0.0006 | 2.86 | 0.0011 | 3.05 | 0.0028 | 2.68 |
| MAST1 | 0.0232 | 2.75 | | | 0.0299 | 3.02 |
| ATP100 | 0.0067 | 2.68 | 0.0036 | 3.51 | | |
| CACNA2D1 | 0.0045 | 2.64 | 0.0031 | 3.29 | 0.0374 | 2.12 |
| ELMO1 | 0.0004 | 2.56 | 0.0006 | 2.80 | 0.0026 | 2.34 |
| UTS2R | 0.0002 | 2.54 | 0.0008 | 2.44 | 0.0004 | 2.64 |
| SCN3A | 0.0015 | 2.53 | 0.0068 | 2.36 | 0.0025 | 2.72 |
| SYP | 0.0226 | 2.45 | | | 0.0197 | 2.90 |
| GABRB3 | 0.0078 | 2.43 | 0.0096 | 2.70 | 0.0311 | 2.20 |
| 6230400G14RIK | 0.0015 | 2.43 | 0.0035 | 2.49 | 0.0050 | 2.37 |
| (Prickle2) | | | | | | |
| SYT13 | 0.0013 | 2.42 | 0.0121 | 2.06 | 0.0011 | 2.83 |
| RAB15 | 0.0058 | 2.40 | 0.0217 | 2.26 | 0.0107 | 2.55 |
| SEZ6L2 | 0.0239 | 2.34 | | | 0.0297 | 2.55 |
| PTPRU | 0.0090 | 2.30 | | | 0.0048 | 2.90 |
| OPN3 | 0.0005 | 2.25 | 0.0031 | 2.10 | 0.0009 | 2.41 |
| SYNGR3 | 0.0256 | 2.24 | 0.0803 | 2.01 | 0.0278 | 2.49 |
| GPR43 | 0.0365 | 2.21 | | | 0.0348 | 2.53 |
| ALOX5AP | 0.0016 | 2.19 | 0.0053 | 2.09 | 0.0034 | 2.20 |
| RAB3A | 0.0282 | 2.12 | | | 0.0458 | 2.17 |
| CHRNAS | 0.0033 | 2.11 | 0.0005 | 3.08 | | |
| KCNG4 | 0.0032 | 2.08 | 0.0089 | 2.05 | 0.0070 | 2.11 |
| CRG-L1 | 0.0016 | 2.04 | 0.0017 | 2.27 | 0.0119 | 1.83 |

### Example 2

### Confirmation of Expression of GUCY2C in mDA Neurons

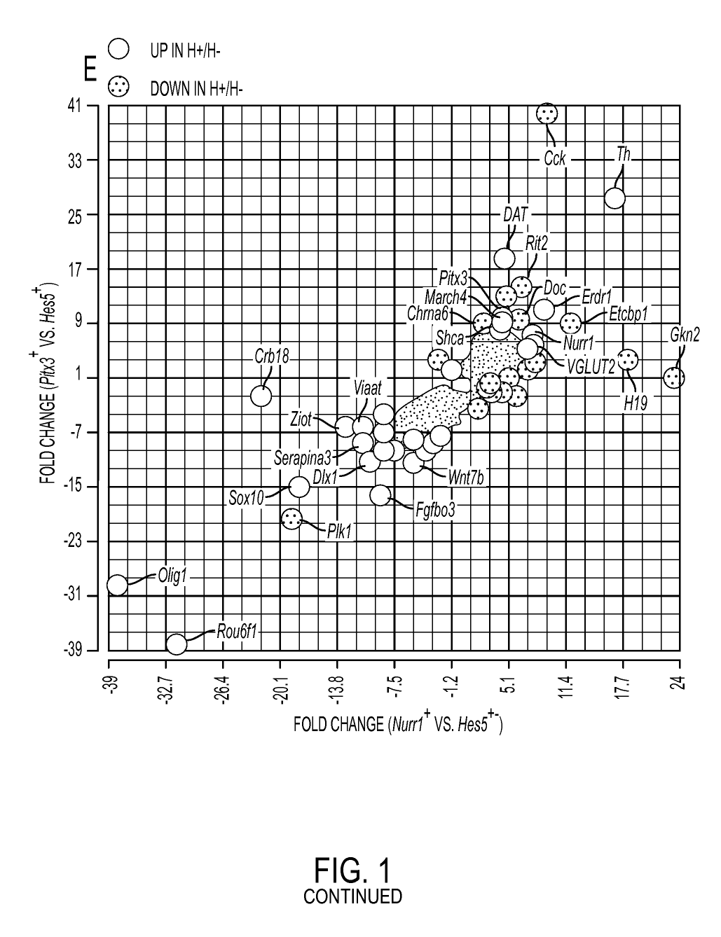

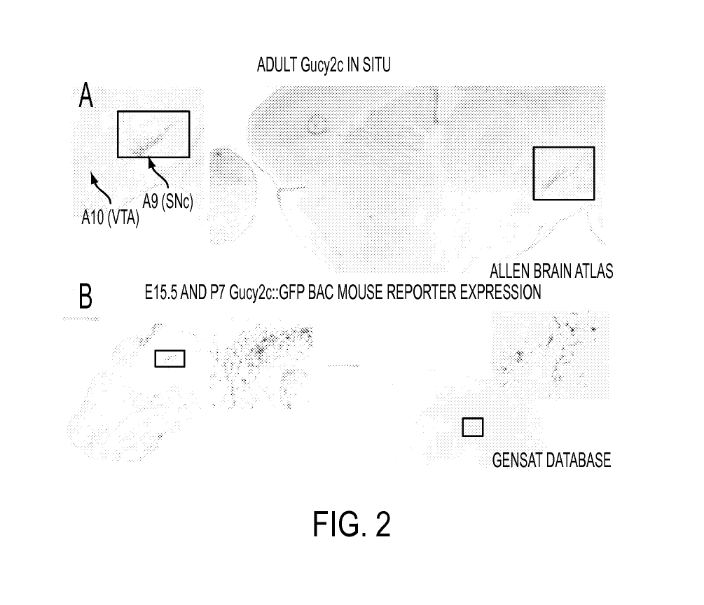

The specific expression of GUCY2C to mDA neurons was confirmed in two separate online mouse gene expression databases. In the Allen brain atlas, GUCY2C mRNA expression was specific to A9 neurons of the adult midbrain as observed by in situ analysis (FIG. 2A). This is significant, because the A9 region, or substantia nigra pars compacta (SNc), contains mDA neurons that are the most affected population of cells in Parkinson's disease and are believed to be responsible for the motor behavior deficits observed in patients; whereas A10, or ventral tegmental area (VTA) is in large part spared from the disease. Because the GUCY2C receptor is not expressed elsewhere in the brain, and is specific to the A9/SNc region, it can be used as a target for both cell sorting experiments, as well as ligand binding assays. The GENSAT database at Rockefeller University tests the expression of BAC reporters for various genes throughout development, and the GUCY2C reporter demonstrated midbrain specific expression at E15.5 and postnatal day 7 (P7) (FIG. 2B).

In vivo confirmation of GUCY2C to mDA neurons was confirmed using a BAC transgenic reporter mouse (GUCY2C::GFP; GENSAT). Sagittal brain section of the e15.5 GUCY2C::GFP BAC reporter mouse demonstrated specific localization of GFP reporter and GUCY2C protein to SN with projections extending into the striatum. These projections also expressed tyrosine hydroxylase, an enzymatic marker of mDA neurons. The BAC reporter (GFP), however, was inefficient at reporting for GUCY2C protein in adult mouse SN, despite correct spactiotemporal expression. Thus, despite specific spatiotemporal expression, the reporter appeared to underreport native GUCY2C protein expression, especially in later stages of development.

### Example 3

### Targeting GUCY2C Receptor Using the Ligand Guanylin

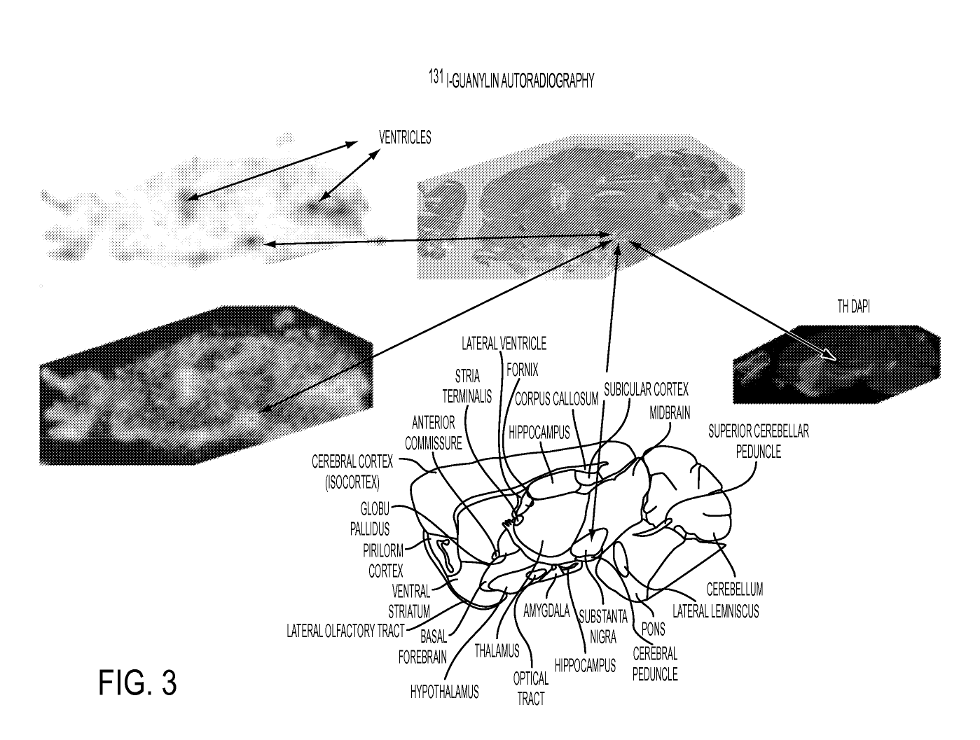

The GUCY2C receptor binds to heat stable enterotoxins, to guanylin and to uroguanylin. Guanylin is a 15 amino acid polypeptide (PGTCEICAYAACTGC, SEQ ID NO:1) that contains a tyrosine residue within the middle of its structure (Bryant et al., Life Sci. 86:760-765 (2010)). Guanylin (R&D Systems, Inc., Minneapolis, Minn.) was radiolabeled with ¹³¹Iodine. 1 mCi of ¹³¹I-guanylin was injected in the tail vein of adult mice and animals were sacrificed 10 minutes post-injection without perfusion. Analysis of sagittal brain sections revealed that ¹³¹I-guanylin localized to the ventricles (which is to be expected as this was an acute injection without perfusion), and within the brain parenchyma the only “hot spot” was indeed localized to the GUCY2C expression domain (FIG. 3, top panel). This signal was shown to be specific to TH-expressing mDA neurons (FIG. 3, top right panel), demonstrating that ¹³¹I-guanylin crossed the blood brain barrier, bound to GUCY2C expressing mDA neurons in the SN of the adult brain, and produced a radioactive signal.

### Example 4

### PET Imaging of ¹²⁴I-Guanylin in Rats

Guanylin is labeled with ¹²⁴I. About 37 MBq of ¹²⁴I-guanylin is dissolved in 0.3 mL of buffered saline and is injected into the tail vein of conscious adult male Sprague-Dawley rats. These animals are then anaesthetized with isoflurane and analysis is performed using a small animal PET scanner at four different time points: 10 minutes, 1 hour, 6 hours and 24 hours (n=4 for each time point), and are compared to saline injected controls. All experiments are carried out under humane conditions with approval from IACUC.

Ex vivo brain autoradiography is also performed in these animals after the PET scan using 50 μm thick coronal brain cryosections of animals that are either perfused with (PBS, 4% paraformaldehyde, and 30% sucrose; n=2) or non-perfused (n=2) flash-frozen brains from each time point. Results from the live PET scans are compared to ex vivo results to determine a time course for ¹²⁴I-guanylin emission and if the ligand can be flushed out during the perfusion process.

Additionally or alternatively, if a signal is determined to be specific to the midbrain, repeat experiments are performed in 6-OHDA (a mDA neuron-specific neurotoxin) lesioned hemiparkinsonian rats as described in, e.g., Ganat et al., J. Clin. Invest. doi: 10.1172/JCI58767 (2012). This unilateral loss of mDA neurons produces a PET signal on one side of the midbrain and is used to determine the binding specificity of ¹²⁴I-guanylin.

### Example 5

### GUCY2C Expression in Human Brain

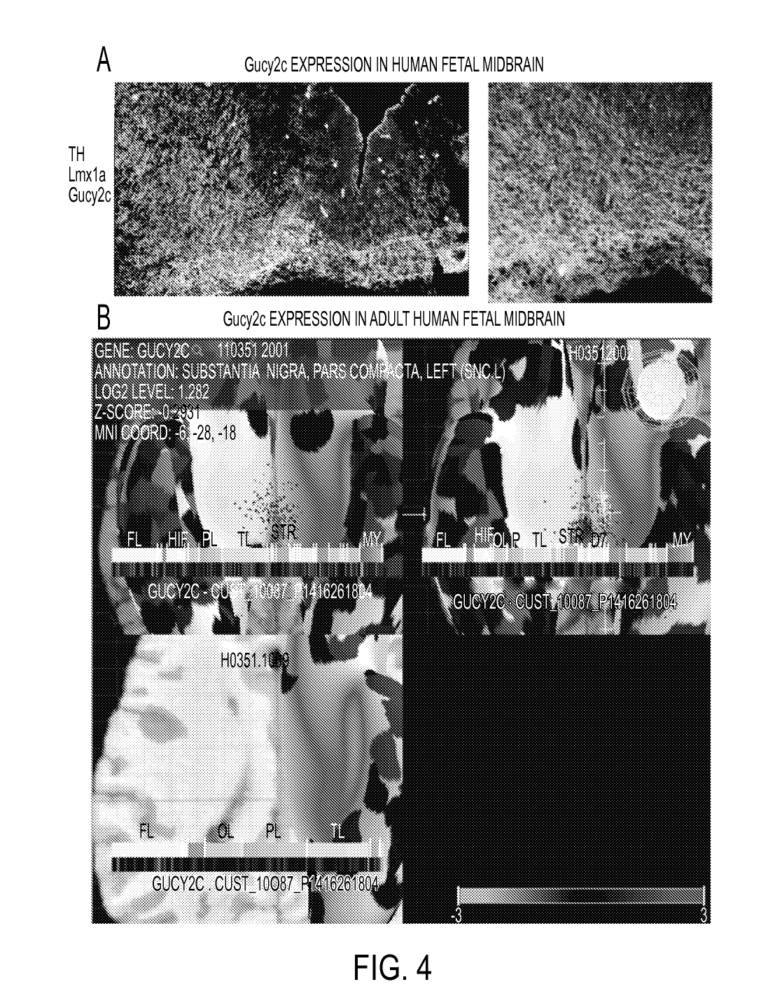

6-8 week human fetal midbrain tissue was analyzed for GUCY2C expression by immunohistochemistry. The results demonstrated that GUCY2C was expressed throughout the Lmx1a+/TH+ mDA neuron precursor domain, as well as into the region of the SN to which these cells migrate and mature into dopamine producing neurons (FIG. 4A). Microarray data of GUCY2C mRNA expression in the Allen brain atlas (http://www.brain-map.org/) indicated that GUCY2C is expressed in the adult SN (FIG. 4B).

### Example 6

### Delivery of ¹²⁴I-Guanylin to Non-Human Primate

The ¹²⁴I-guanylin radiotracer is prepared and healthy adult rhesus monkeys are anaesthetised with ketamine-xylazine before and during the procedure under the following protocol: 10 mg/kg ketamine plus 0.5 mg/kg xylazine, approximately 1 h before tracer injection, followed by 5 mg/kg ketamine plus 0.2 mg/kg xylazine immediately after tracer injection. PET studies are performed using a GE advance tomograph. The animal is injected with approximately 37 MBq of ¹²⁴I-guanylin. Images are acquired according to the following protocol: starting immediately after the injection of the radiotracer five 2-min scans are obtained, followed by ten 5-min scans for a total sampling time of 1 hour. Circular regions of interest are drawn on SN, cerebellum and cortex bilaterally using Analyze software.

¹²⁴I-guanylin radioactivity concentration in plasma is also evaluated in an additional subject. In brief, approximately 1 ml of blood is collected at 1, 5, 10, 20, 30 and 90 min and centrifuged, and approximately 100 μl of plasma is counted in a gamma counter. The remaining plasma of samples collected at 5, 10, 20 and 30 min are extracted with CH₃CN (1:1 vol/vol) and analyzed for the presence of radioactive metabolites.

Additionally or alternatively, midbrain-specific PET signals are determined in MPTP-treated hemiparkinsonian monkeys. MPTP is a neurotoxin known to selectively kill mDA neurons in the SN, and has been used to generate Parkinsonian motor deficits in non-human primates. Hemiparkinsonian monkeys are generated as described in Mondal et al., J. Neuroimmune Pharmacol. DOI: 10.1007/s11481-012-9377-9 (2012). Briefly, 6-8 year old rhesus monkeys animals are tranquilized with ketamine (10 mg/kg, i.m.) and maintained on an anesthetic plane with isoflurane (1-2%). For each MPTP injection, a right-sided incision is made along the medial edge of the sternocleidomastoid muscle. A 27 gauge butterfly needle is inserted into the common carotid artery in a direction retrograde to blood flow; for each injection, 20 ml of saline containing 3 mg of MPTP-HCl (Sigma) is infused at a rate of 1.33 ml/min for 15 min. After the infusion is completed, 3 ml of saline is delivered, and then the incision is closed.

Monkeys are trained to a consistent level of performance before and after the intracarotid injection of MPTP, and are subsequently scored for their performance using a unified PD rating scale (UPDRS) that measures 10 parkinsonian features: tremor, posture, locomotion, hypokinesia, bradykinesia, balance, fine and gross motor skills, startle response, and freezing. A monkey that displays hemiparkinsonian deficits, as well as a non-treated healthy control is injected with ¹²⁴I-guanylin and the PET signal is analyzed as previously described. Additionally, postmortem immunohistochemical analysis of SN sections is performed to determine the number of TH+ cells in both MPTP and non-MPTP treated monkeys. This allows quantification of a PET signal-to-cell number ratio.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a GUCY2C receptor ligand conjugate comprising a GUCY2C receptor ligand conjugated to a payload moiety, wherein upon administration of the composition to a subject, the GUCY2C receptor ligand conjugate crosses the blood brain barrier of the subject.

2. The composition of claim 1, wherein the GUCY2C receptor ligand conjugate consists of a GUCY2C receptor ligand conjugated to a payload moiety, wherein upon administration of the composition to a subject, the GUCY2C receptor ligand conjugate crosses the blood brain barrier of the subject.

3. The composition of claim 1, wherein the GUCY2C receptor ligand conjugate, when placed in an in vitro endothelial cell model of the BBB, at least about 50% crosses the model BBB.

4. The composition of claim 1, wherein the payload moiety is a detectable moiety.

5. The composition of claim 4, wherein the detectable moiety is a radioactive label or a fluorescent label.

6. The composition of claim 1, wherein the payload moiety is a therapeutic moiety.

7. The composition of claim 6, wherein the therapeutic moiety is a neuroprotective agent.

8. The composition of claim 6, wherein the neuroprotective agent is L-dopa.

9-10. (canceled)

11. A method of treating a subject for Parkinson's disease, the method comprising:
administering to a subject a GUCY2C receptor ligand conjugate comprising a GUCY2C receptor ligand conjugated to a detectable moiety, wherein the GUCY2C receptor ligand conjugate crosses the blood brain barrier of the subject;
measuring a level of the detectable moiety bound to neurons in the midbrain of the subject; and
administering a therapeutic agent to the subject if the level of detectable moiety bound to neurons is less than a predetermined level.

12. The method of claim 11, wherein the therapeutic agent is a neuroprotective agent.

13. The method of claim 12, wherein the neuroprotective agent is L-dopa.

14. A method of treating a subject having or at risk of developing Parkinson's disease, comprising:
administering to the subject a GUCY2C receptor ligand conjugate comprising a GUCY2C receptor ligand conjugated to a therapeutic moiety, wherein the GUCY2C receptor ligand conjugate crosses the blood brain barrier of the subject, thereby treating the subject.

15. The method of claim 14, wherein the GUCY2C receptor ligand conjugate specifically binds to neurons in the midbrain of the subject.

16. (canceled)

17. The method of claim 14, wherein the therapeutic moiety comprises a neuroprotective agent.

18. The method of claim 17, wherein the neuroprotective agent is L-dopa.

19. (canceled)

20. The composition of claim 1, wherein the GUCY2C receptor ligand is guanylin.

21. The composition of claim 4, wherein the detectable moiety is ¹²⁴I.

22. The method of claim 11, wherein the GUCY2C receptor ligand is guanylin.

23. The method of claim 11, wherein the detectable moiety is ¹²⁴I.

24. The method of claim 23, wherein measuring the level of the detectable moiety comprises positron emission tomography (PET).


SEQUENCE LISTING (2 sequences)

SEQ ID NO: 1 - protein, 15 aa, Homo sapiens
PNTCEICAYAACTGC

SEQ ID NO: 2 - protein, 15 aa, Artificial Sequence
  Chemically syntehsized polypeptide Uroguanylin(GUCA2B)
QEDCELCINVACTGC